(19) United States
(12) Patent Application Publication
(10) Pub. No.: US 2006/0241700 A1
(43) Pub. Date: Oct. 26, 2006

(54) Level crossing detector for detecting noise, sinus rhythm and ventricular fibrillation in subcutaneous or body surface signals

(76) Inventors: Raja N. Ghanem, Minneapolis, MN (US); Jeffrey M. Gillberg, Coon Rapids, MN (US)

Correspondence Address: MEDTRONIC, INC., 710 MEDTRONIC PARK, MINNEAPOLIS, MN, 55432-9924, US

(21) Appl. No.: 11/114,462

(22) Filed: Apr. 26, 2005

(51) Int. Cl. A61N 1/39

(52) U.S. Cl. 607/5

(57) ABSTRACT

A SubQ ICD that is entirely implantable subcutaneously with minimal surgical intrusion into the body of the patient and associated with subcutaneous leads provides distributed cardioversion-defibrillation sense and stimulation electrodes for delivery of cardioversion-defibrillation shock and pacing therapies across the heart when necessary. A level crossing detection system and process is implemented to detect noise, sinus rhythm and ventricular fibrillation in subcutaneous or body surface signals to deliver therapies as needed.

### FIELD OF THE INVENTION

The present invention generally relates to an implantable medical device system, particularly a subcutaneous ICD (SubQ ICD) and an improved detection system and method for detecting arrhythmias from sinus tachycardia and noise in subcutaneous ECG signals.

### BACKGROUND OF THE INVENTION

Many types of implantable medical devices (IMDs) have been clinically implanted over the last twenty years that deliver relatively high-energy cardioversion and/or defibrillation shocks to a patient's heart when a malignant tachyarrhythmia, e.g., atrial or ventricular fibrillation, is detected. Cardioversion shocks are typically delivered in synchrony with a detected R-wave when fibrillation detection criteria are met, whereas defibrillation shocks are typically delivered when fibrillation criteria are met and an R-wave cannot be discerned from the electrogram (EGM).

The current state of the art of ICDs or implantable pacemaker/cardioverter/defibrillators (PCDs) includes a full featured set of extensive programmable parameters which includes multiple arrhythmia detection criteria, multiple therapy prescriptions (for example, stimulation for pacing in the atrial, ventricular and dual chamber; atrial and ventricular for bradycardia; bi-atrial and/or bi-ventricular for heart failure; and arrhythmia overdrive or entrainment stimulation; and high level stimulation for cardioversion and/or defibrillation), extensive diagnostic capabilities and high speed telemetry systems. These full-featured ICDs or PCDs, hereinafter IMD, are typically implanted into patients who have had, and survived, a significant cardiac event (such as sudden death). Additionally, these devices are expected to last up to 5-8 years and/or provide at least 200 life saving therapy episodes.

Even though there have been great strides in size reduction over the past 20 years, the incorporation of all these features in an IMD, including the longevity requirements, dictates that the devices be typically much larger than current state of the art pacemakers. Such devices are often difficult to implant in some patients (particularly children and thin, elderly patients) and typically require the sacrifice of 1 or 2 veins to implant the lead system. Current technology for the implantation of an IMD uses a transvenous approach for cardiac electrodes and lead wires. The defibrillator canister/housing is generally implanted as an active can for defibrillation and electrodes positioned in the heart are used for pacing, sensing and detection of arrhythmias.

Although IMDs and implant procedures are very expensive, most patients who are implanted have experienced and survived a sudden cardiac death episode because of interventional therapies delivered by the IMDs. Survivors of sudden cardiac death episodes are in the minority, and studies are ongoing to identify patients who are asymptomatic by conventional measures but are nevertheless at risk of a future sudden death episode. Current studies of patient populations, e.g., the MADIT II and SCDHeFT studies, are establishing that there are large numbers of patients in any given population that are susceptible to sudden cardiac death, that they can be identified with some degree of certainty and that they are candidates for a prophylactic implantation of a defibrillator (often called primary prevention). However, implanting currently available IMDs in all such patients would be prohibitively expensive. Further, even if the cost factor is eliminated there is shortage of trained personnel and implanting resources.

One option proposed for this patient population is to implant a prophylactic subcutaneous implantable cardioverter/defibrillator (SubQ ICD) such that when these patients receive a shock and survived a cardiac episode, they will ultimately have an implant with a full-featured ICD and transvenous leads.

While there are a few small populations in whom SubQ ICD might be the first choice of implantation for a defibrillator, the vast majority of patients are physically suited to be implanted with either an ICD or SubQ ICD. It is likely that pricing of the SubQ ICD will be at a lower price point than an ICD. Further, as SubQ ICD technology evolves, it may develop a clear and distinct advantage over ICDs. For example, the SubO ICD does not require leads to be placed in the bloodstream. Accordingly, complications arising from leads placed in the cardiovasculature environment is eliminated. Further, endocardial lead placement is not possible with patients who have a mechanical heart valve implant and is not generally recommended for pediatric cardiac patients. For these and other reasons, a SubQ ICD may be preferred over an ICD.

There are technical challenges associated with the implantation of a SubQ ICD. For example, SubQ ICD sensing is challenged by the presence of muscle artifact, respiration and other physiological signal sources. This is particularly because the SubQ ICD is limited to far-field sensing since there are no intracardial or epicardial electrodes in a subcutaneous system. Further, sensing of atrial activation from subcutaneous electrodes is limited since the atria represent a small muscle mass and the atrial signals are not sufficiently detectable transthoracically. Thus, SubO ICD sensing presents a bigger challenge than an ICD which has the advantage of electrodes inside the heart and, especially, inside the atrium. Accordingly, the design of a SubQ ICD is a difficult proposition given the technical challenges to sense and detect arrhythmias.

Yet another challenge could be combining a SubQ ICD with an existing pacemaker (IPG) in a patient. While this may be desirable in a case where an IPG patient may need a defibrillator, a combination implant of SubQ ICD and IPG may result in inappropriate therapy by the SubQ ICD, which may pace or shock based on spikes from the IPG. Specifically, each time the IPG emits a pacing stimulus, the SubQ ICD may interpret it as a genuine cardiac beat. The result can be over-counting beats from the atrium, ventricles or both; or, because of the larger pacing spikes, sensing of arrhythmic signals (which are typically much smaller in amplitude) may be compromised.

Thus, providing a robust detection in the presence of challenges presented by SubQ ICD requirements and the environment under which it is expected to perform calls for special considerations.

Therefore, for these and other reasons, a need exists for an improved method and apparatus to reliably sense and detect arrhythmias, subcutaneously, while rejecting noise and other physiologic signals.

### SUMMARY OF THE INVENTION

A method and apparatus is described which provides for an improved detection of arrhythmias via an ECG signal obtained from a SubQ ICD, with no endocardial or epicardial leads. Specifically, the invention includes utilizing signal crossings within a fixed time window with a threshold signal based on subcutaneous signal characteristics and subsequent generation and evaluation of a histogram to distinguish between noise, sinus rhythm and ventricular fibrillation.

### BRIEF DESCRIPTION OF THE DRAWINGS

These and other features of the present invention will be appreciated as the same becomes better understood by reference to the following detailed description of the embodiments of the invention when considered in connection with the accompanying drawings, in which like numbered reference numbers designate like parts throughout the figures thereof, and wherein:

FIG. 1 depicts a SubQ ICD implanted in a patient;

FIG. 2 depicts a frontal and side view of a SubQ ICD and an electrical lead body associated therewith;

FIG. 3 is a circuit diagram of an embodiment of the circuitry of the SubQ ICD in accordance with the present invention.

FIG. 4 is a block diagram representing the sensing circuitry of the SubQ ICD in accordance with the present invention.

FIG. 5a is a representation of signals for normal sinus rhythm derived from a subcutaneous ECG signal;

FIG. 5b is a representation of signals for ventricular fibrillation derived from a subcutaneous ECG signal;

FIG. 5c is a representation of noise signals derived from a subcutaneous ECG signal; and

FIG. 6 is a simplified flow diagram illustrating the method of detection of arrhythmias by the SubQ ICD in accordance with the present invention.

### DETAILED DESCRIPTION OF THE INVENTION

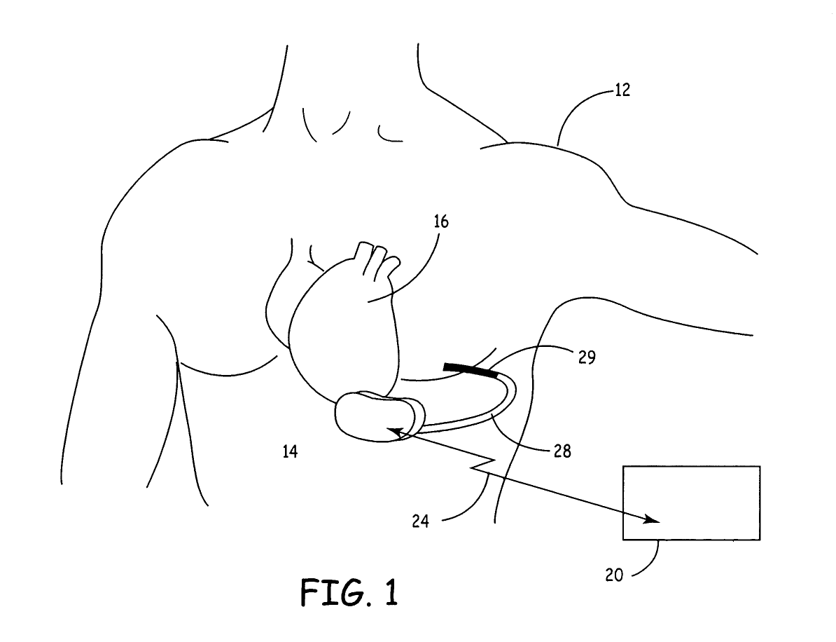

FIG. 1 shows SubQ ICD 14 implanted in patient 12. The SubQ ICD 14 is subcutaneously implanted outside the ribcage of patient 12, anterior to the cardiac notch. Further, a subcutaneous sensing and cardioversion/defibrillation therapy delivery lead 28 in electrical communication with SubQ ICD 14, is tunneled subcutaneously into a location adjacent to a portion of a latissimus dorsi muscle of patient 12. Specifically, lead 28 is tunneled subcutaneously from the median implant pocket of SubQ ICD 14 laterally and posterially to the patient's back to a location opposite the heart such that the heart 16 is disposed between the SubQ ICD 14 and the distal electrode coil 29 of lead 28.

Further referring to FIG. 1, programmer 20 is shown in telemetric communication with SubQ ICD 14 by RF communication link 24 such as Bluetooth, WiFi, MICS, or as described in U.S. Pat. No. 5,683,432 “Adaptive Performance-Optimizing Communication System for Communicating with an Implantable Medical Device” to Goedeke, et al and incorporated herein by reference in its entirety.

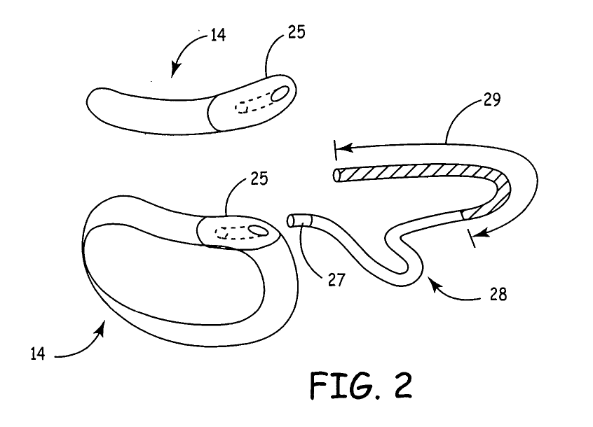

FIG. 2 is a frontal and plan view of SubQ ICD 14. SubO ICD 14 is an ovoid and includes a substantially kidney-shaped profile forming a housing with connector 25 for attaching a subcutaneous sensing and cardioversion/defibrillation therapy delivery lead 28. SubQ ICD 14 may be constructed of stainless steel, titanium or ceramic as described in U.S. Pat. Nos. 4,180,078 “Lead Connector for a Body Implantable Stimulator” to Anderson and 5,470,345 “Implantable Medical Device with Multi-layered Ceramic Enclosure” to Hassler, et al. The electronics circuitry of SubQ ICD 14 may be incorporated on a polyamide flex circuit, printed circuit board (PCB) or ceramic substrate with integrated circuits packaged in leadless chip carriers and/or chip scale packaging (CSP). The plan view shows the ovoid construction that promotes ease of subcutaneous implant. This structure is ergonomically adapted to minimize patient discomfort during normal body movement and flexing of the thoracic musculature.

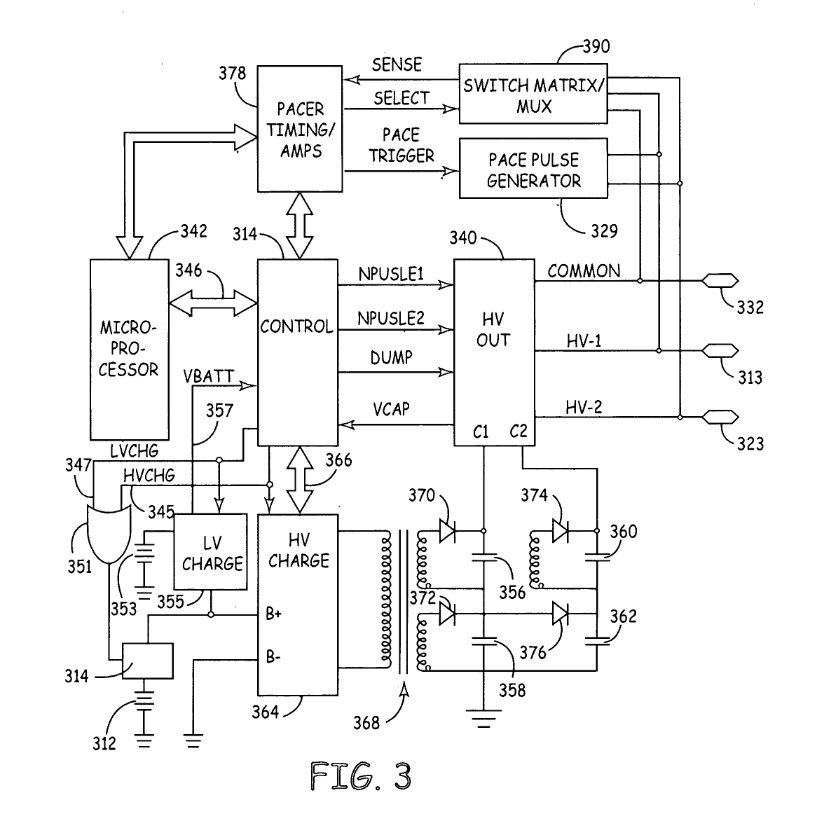

The electronic circuitry employed in SubQ ICD 14 can take any of the known forms that detect a tachyarrhythmia from the sensed ECG and provide cardioversion/defibrillation shocks as well as post-shock pacing as needed while the heart recovers. A simplified block diagram of such circuitry adapted to function employing the first and second and, optionally, the third cardioversion-defibrillation electrodes as well as the ECG sensing and pacing electrodes described herein below is set forth in FIG. 3. It will be understood that the simplified block diagram does not show all of the conventional components and circuitry of such ICDs including digital clocks and clock lines, low voltage power supply and supply lines for powering the circuits and providing pacing pulses or telemetry circuits for telemetry transmissions between the SubQ ICD 14 and external programmer 20.

FIG. 3 depicts the electronic circuitry including low voltage and high voltage batteries within the hermetically sealed housing of SubQ ICD 14. The low voltage battery 353 is coupled to a power supply (not shown) that supplies power to the SubQ ICD 14 circuitry and the pacing output capacitors to supply pacing energy in a manner well known in the art. The low voltage battery can comprise one or two conventional LiCFₓ, LiMnO₂ or Lil₂ cells. The high voltage battery 312 can comprise one or two conventional LiSVO or LiMnO₂ cell.

Further referring to FIG. 3, SubQ ICD 14 functions are controlled by means of software, firmware and hardware that cooperatively monitor the ECG, determine when a cardioversion-defibrillation shock or pacing is necessary, and deliver prescribed cardioversion-defibrillation and pacing therapies. The block diagram of FIG. 3 incorporates circuitry set forth in commonly assigned U.S. Pat. Nos. 5,163,427 “Apparatus for Delivering Single and Multiple Cardioversion and Defibrillation Pulses” to Keimel and 5,188,105 “Apparatus and Method for Treating a Tachyarrhythmia” to Keimel for selectively delivering single phase, simultaneous biphasic and sequential biphasic cardioversion-defibrillation shocks typically employing an ICD IPG housing electrode coupled to the COMMON output 312 of high voltage output circuit 340 and one or two cardioversion-defibrillation electrodes disposed posterially and subcutaneously and coupled to the HVI and HV-2 outputs (313 and 323, respectively) of the high voltage output circuit 340. The circuitry of the SubQ ICD 14 of the present invention could be simplified by adoption of one such cardioversion-defibrillation shock waveform for delivery between the first and second cardioversion-defibrillation electrodes 313 and 323 coupled to the HV-I and HV-2 outputs respectively. Alternatively, the third cardioversion-defibrillation electrode 332 can be coupled to the COMMON output as shown in FIG. 3 and the first and second cardioversion-defibrillation electrodes 313 and 323 can be electrically connected in to the HV-I and the HV-2 outputs, respectively, as depicted in FIG. 3.

The cardioversion-defibrillation shock energy and capacitor charge voltages can be intermediate to those supplied by ICDs having at least one cardioversion-defibrillation electrode in contact with the heart and most AEDs having cardioversion-defibrillation electrodes in contact with the skin. The typical maximum voltage necessary for ICDs using most biphasic waveforms is approximately 750 Volts with an associated maximum energy of approximately 40 Joules. The typical maximum voltage necessary for AEDs is approximately 2000-5000 Volts with an associated maximum energy of approximately 200-360 Joules depending upon the model and waveform used. The SubQ ICD of the present invention uses maximum voltages in the range of about 700 to about 3150 Volts and is associated with energies of about 25 Joules to about 210 Joules. The total high voltage capacitance could range from about 50 to about 300 microfarads. Such cardioversion-defibrillation shocks are only delivered when a malignant tachyarrhythmia, e.g., ventricular fibrillation is detected through processing of the far field cardiac ECG employing one of the available detection algorithms known in the ICD art.

In FIG. 3, pacer timing/sense amplifier circuit 378 processes the far field ECG SENSE signal that is developed across a particular ECG sense vector defined by a selected pair of the electrodes 332, 313 and, optionally, electrode 323 if present as noted above. The selection of the sensing electrode pair is made through the switch matrix/MUX 390 in a manner to provide the most reliable sensing of the EGM signal of interest, which would be the R wave for patients who are believed to be at risk of ventricular fibrillation leading to sudden death. The far field ECG signals are passed through the switch matrix/MUX 390 to the input of a sense amplifier in the pacer timing/sense amplifier circuit 378. Bradycardia is typically determined by an escape interval timer within the pacer timing circuit 378 or the timing and control circuit 344, and pacing pulses that develop a PACE TRIGGER signal applied to the pacing pulse generator 392 when the interval between successive R-waves exceeds the escape interval. Bradycardia pacing is often temporarily provided to maintain cardiac output after delivery of a cardioversion-defibrillation shock that may cause the heart to slowly beat as it recovers back to normal function.

Detection of a malignant tachyarrhythmia is determined in the timing and control circuit 344 as a function of the intervals between R-wave sense event signals that are output from the pacer timing/sense amplifier circuit 378 to the timing and control circuit 344. It should be noted that the present invention utilizes not only the interval based signal analysis method but also the histogram signal processing method and apparatus as described hereinbelow.

Certain steps in the performance of the detection algorithm criteria are cooperatively performed in microcomputer 342, including microprocessor, RAM and ROM, associated circuitry, and stored detection criteria that may be programmed into RAM via a telemetry interface (not shown) conventional in the art. Data and commands are exchanged between microcomputer 342 and timing and control circuit 344, pacer timing/amplifier circuit 378, and high voltage output circuit 340 via a bidirectional data/control bus 346. The pacer timing/amplifier circuit 378 and the timing and control circuit 344 are clocked at a slow clock rate. The microcomputer 342 is normally asleep, but is awakened and operated by a fast clock by interrupts developed by each R-wave sense event or on receipt of a downlink telemetry programming instruction or upon delivery of cardiac pacing pulses to perform any necessary mathematical calculations, to perform tachycardia and fibrillation detection procedures, and to update the time intervals monitored and controlled by the timers in pace/sense circuitry 378.

The algorithms and functions of the microcomputer 342 and timer and control circuit 344 employed and performed in detection of tachyarrhythmias are set forth, for example, in commonly assigned U.S. Pat. Nos. 5,354,316 “Method and Apparatus for Detection and Treatment of Tachycardia and Fibrillation” to Keimel; 5,545,186 “Prioritized Rule Based Method and Apparatus for Diagnosis and Treatment of Arrhythmias” to Olson, et al, 5,855,593 “Prioritized Rule Based Method and Apparatus for Diagnosis and Treatment of Arrhythmias” to Olson, et al and 5,193,535 “Method and Apparatus for Discrimination of Ventricular Tachycardia from Ventricular Fibrillation and Treatment Thereof” to Bardy, et al, (all incorporated herein by reference in their entireties). Particular algorithms for detection of ventricular fibrillation and malignant ventricular tachycardias can be selected from among the comprehensive algorithms for distinguishing atrial and ventricular tachyarrhythmias from one another and from high rate sinus rhythms that are set forth in the '316, '186, '593 and '593 patents.

The detection algorithms are highly sensitive and specific for the presence or absence of life threatening ventricular arrhythmias, e.g., ventricular tachycardia (V-TACH) and ventricular fibrillation (V-FIB). Another optional aspect of the present invention is that the operational circuitry can detect the presence of atrial fibrillation (A FIB) as described in Olson, W. et al. “Onset And Stability For Ventricular Tachyarrhythmia Detection in an Implantable Cardioverter and Defibrillator,” Computers in Cardiology (1986) pp. 167-170. Detection can be provided via R-R Cycle length instability detection algorithms. Once A-FIB has been detected, the operational circuitry will then provide QRS synchronized atrial cardioversion/defibrillation using the same shock energy and wave shapes used for ventricular cardioversion/defibrillation.

Operating modes and parameters of the detection algorithm are programmable and the algorithm is focused on the detection of V-FIB and high rate V-TACH (>180 bpm). Although SubQ ICD 14 of the present invention may rarely be used for an actual sudden death event, the simplicity of design and implementation allows it to be employed in large populations of patients at relatively little or no risk with modest cost by medical personnel other than electrophysiologists. Consequently, SubQ ICD 14 of the present invention includes the automatic detection and therapy of the most malignant rhythm disorders. As part of the detection algorithm's applicability to children, the upper rate range is programmable upward for use in children, known to have rapid supraventricular tachycardias and more rapid V-FIB.

When a malignant tachycardia is detected, high voltage capacitors 356, 358, 360, and 362 are charged to a pre-programmed voltage level by a high-voltage charging circuit 364. It is generally considered inefficient to maintain a constant charge on the high voltage output capacitors 356, 358, 360, 362. Instead, charging is initiated when control circuit 344 issues a high voltage charge command HVCHG delivered on line 345 to high voltage charge circuit 364 and charging is controlled by means of bi-directional control/data bus 366 and a feedback signal VCAP from the HV output circuit 340. High voltage output capacitors 356, 358, 360 and 362 may be of film, aluminum electrolytic or wet tantalum construction.

The negative terminal of high voltage battery 312 is directly coupled to system ground. Switch circuit 314 is normally open so that the positive terminal of high voltage battery 312 is disconnected from the positive power input of the high voltage charge circuit 364. The high voltage charge command HVCHG is also conducted via conductor 349 to the control input of switch circuit 314, and switch circuit 314 closes in response to connect positive high voltage battery voltage EXT B+ to the positive power input of high voltage charge circuit 364. Switch circuit 314 may be, for example, a field effect transistor (FET) with its source-to-drain path interrupting the EXT B+ conductor 318 and its gate receiving the HVCHG signal on conductor 345. High voltage charge circuit 364 is thereby rendered ready to begin charging the high voltage output capacitors 356, 358, 360, and 362 with charging current from high voltage battery 312.

High voltage output capacitors 356, 358, 360, and 362 may be charged to very high voltages, e.g., 700-3150V, to be discharged through the body and heart between the selected electrode pairs among first, second, and, optionally, third subcutaneous cardioversion-defibrillation electrodes 313, 323, and 332. The details of the voltage charging circuitry are also not deemed to be critical with regard to practicing the present invention; one high voltage charging circuit believed to be suitable for the purposes of the present invention is disclosed. High voltage capacitors 356, 358, 360, and 362 are charged by high voltage charge circuit 364 and a high frequency, high-voltage transformer 368 as described in detail in commonly assigned U.S. Pat. No. 4,548,209 “Energy Converter for Implantable Cardioverter” to Wielders, et al. Proper charging polarities are maintained by diodes 370, 372, 374 and 376 interconnecting the output windings of high-voltage transformer 368 and the capacitors 356, 358, 360, and 362. As noted above, the state of capacitor charge is monitored by circuitry within the high voltage output circuit 340 that provides a VCAP, feedback signal indicative of the voltage to the timing and control circuit 344. Timing and control circuit 344 terminates the high voltage charge command HVCHG when the VCAP signal matches the programmed capacitor output voltage, i.e., the cardioversion-defibrillation peak shock voltage.

Timing and control circuit 344 then develops first and second control signals NPULSE 1 and NPULSE 2, respectively, that are applied to the high voltage output circuit 340 for triggering the delivery of cardioverting or defibrillating shocks. In particular, the NPULSE 1 signal triggers discharge of the first capacitor bank, comprising capacitors 356 and 358. The NPULSE 2 signal triggers discharge of the first capacitor bank and a second capacitor bank, comprising capacitors 360 and 362. It is possible to select between a plurality of output pulse regimes simply by modifying the number and time order of assertion of the NPULSE 1 and NPULSE 2 signals. The NPULSE 1 signals and NPULSE 2 signals may be provided sequentially, simultaneously or individually. In this way, control circuitry 344 serves to control operation of the high voltage output stage 340, which delivers high energy cardioversion-defibrillation shocks between a selected pair or pairs of the first, second, and, optionally, the third cardioversion-defibrillation electrodes 313, 323, and 332 coupled to the HV-I, HV-2 and optionally to the COMMON output as shown in FIG. 3.

Thus, SubQ ICD 14 monitors the patient's cardiac status and initiates the delivery of a cardioversion-defibrillation shock through a selected pair or pairs of the first, second and third cardioversion-defibrillation electrodes 313, 323 and 332 in response to detection of a tachyarrhythmia requiring cardioversion-defibrillation. The high HVCHG signal causes the high voltage battery 312 to be connected through the switch circuit 314 with the high voltage charge circuit 364 and the charging of output capacitors 356, 358, 360, and 362 to commence. Charging continues until the programmed charge voltage is reflected by the VCAP signal, at which point control and timing circuit 344 sets the HVCHG signal low terminating charging and opening switch circuit 314. Typically, the charging cycle takes only fifteen to twenty seconds, and occurs very infrequently. The SubQ ICD 14 can be programmed to attempt to deliver cardioversion shocks to; the heart in the manners described above in timed synchrony with a detected R-wave or can be programmed or fabricated to deliver defibrillation shocks to the heart in the manners described above without attempting to synchronize the delivery to a detected R-wave. Episode data related to the detection of the tachyarrhythmia and delivery of the cardioversion-defibrillation shock can be stored in RAM for uplink telemetry transmission to an external programmer as is well known in the art to facilitate in diagnosis of the patient's cardiac state. A patient receiving the ICD 10 on a prophylactic basis would be instructed to report each such episode to the attending physician for further evaluation of the patient's condition and assessment for the need for implantation of a more sophisticated and long-lived ICD.

SubO ICD 14 desirably includes telemetry circuit (not shown in FIG. 3), so that it is capable of being programmed by means of external programmer 20 via a 2-way telemetry link 24 (shown in FIG. 1). Uplink telemetry allows device status and diagnostic/event data to be sent to external programmer 20 for review by the patient's physician. Downlink telemetry allows the external programmer via physician control to allow the programming of device function and the optimization of the detection and therapy for a specific patient. Programmers and telemetry systems suitable for use in the practice of the present invention have been well known for many years. Known programmers typically communicate with an implanted device via a bidirectional radio-frequency telemetry link, so that the programmer can transmit control commands and operational parameter values to be received by the implanted device, and so that the implanted device can communicate diagnostic and operational data to the programmer. Programmers believed to be suitable for the purposes of practicing the present invention include the Models 9790 and CareLink® programmers, commercially available from Medtronic; Inc., Minneapolis, Minnesota. Various telemetry systems for providing the necessary communications channels between an external programming unit and an implanted device have been developed and are well known in the art. Telemetry systems believed to be suitable for the purposes of practicing the present invention are disclosed, for example, in the following U.S. Patents: U.S. Pat. No. 5,127,404 to Wyborny et al. entitled “Telemetry Format for Implanted Medical Device”; U.S. Pat. No. 4,374,382 to Markowitz entitled “Marker Channel Telemetry System for a Medical Device”; and U.S. Pat. No. 4,556,063 to Thompson et al. entitled “Telemetry System for a Medical Device”. The Wyborny et al. '404, Markowitz '382, and Thompson et al. '063 patents are commonly assigned to the assignee of the present invention, and are each hereby incorporated by reference herein in their respective entireties.

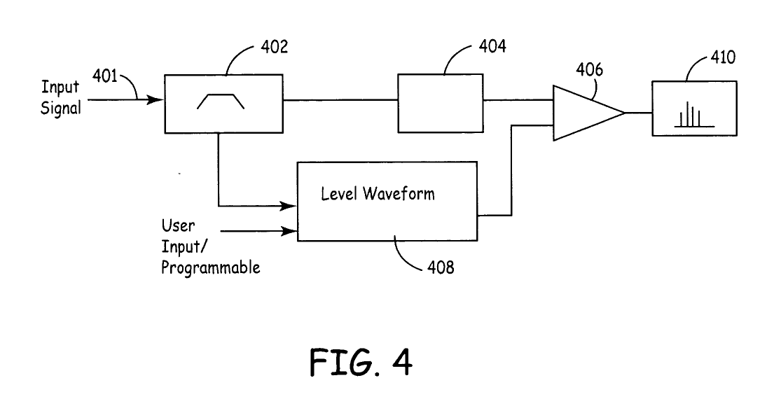

FIG. 4 shows a block diagram 400 relating to the signal processing aspects of the invention The ECG signal 401 from the distal electrode 29 of subcutaneous lead 28 and the electrode on the SubQ ICD 14 is amplified and bandpass filtered (0.67-30 Hz) by amplifier 402 located in Pacer Timing/Amps 378 of FIG. 3. Narrow band amplifier/filter 402 is desirably a finite impulse response filter (FIR). Rectifier block 404 performs full wave rectification on the amplified signal from bandpass filter 402. A level waveform is derived from the narrowband signal at processing block 406. A programmable fixed threshold, a moving average or, alternatively, an auto-adjusting threshold is generated as described in U.S. Pat. No. 5,117,824 “Apparatus for Monitoring Electrical Physiologic Signals” to Keimel, et al incorporated herein by reference in its entirety. A comparator 406 determines signal crossings from the selected and generated threshold from level block 408. Microprocessor 342, control circuit 344 and programs located in RAM/ROM generate a histogram with 10 ms bins, for example, of signal crossings in adjacent two-second windows at block 410. A single or several histograms from adjacent windows are evaluated to allow detection of normal sinus rhythm, physiologic noise signals, sinus tachycardia, ventricular tachycardia and/or ventricular fibrillation. The examples shown are two-second windows, but other window sizes may be used. It is likely that a window size between two and 10 seconds would provide best performance and a desired window size is three seconds. A programmable n of m (i.e., 2 of 3) criteria is used to make the final determination of VF or VT versus non-life-threatening signals.

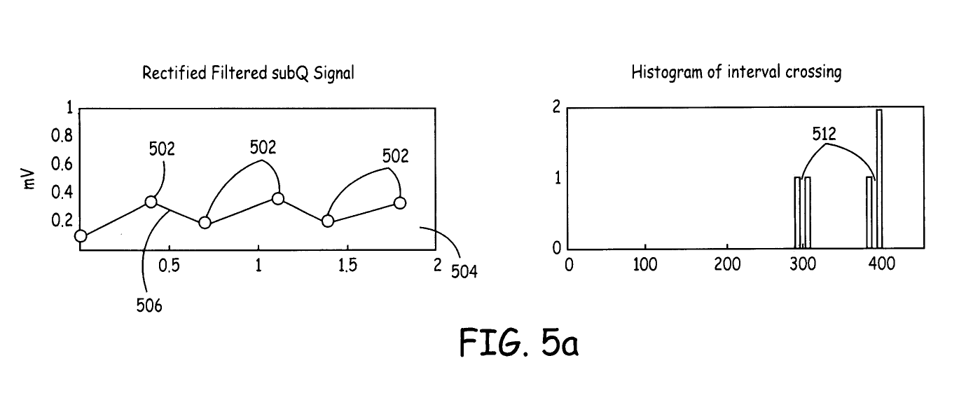

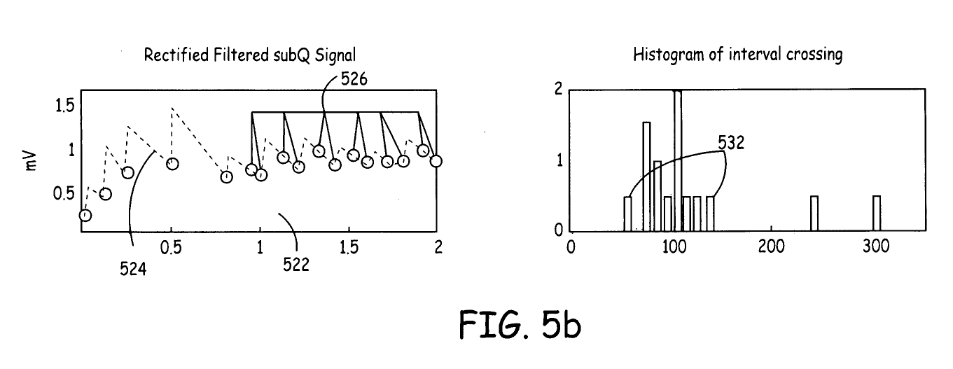

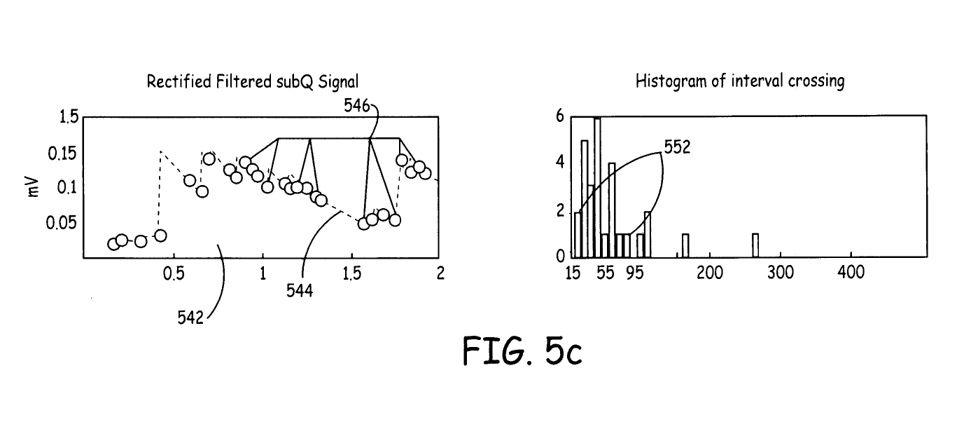

FIG. 5A shows a two-second window 500 with a rectified and filtered subcutaneous normal sinus rhythm signal 502. The auto-adjusting threshold waveform is also shown at 504. Rising edge crossings between the subcutaneous signal and the auto-adjusting threshold are depicted at 506. Histograms of the signal crossings are shown for a two-second window at 510. Note that normal sinus rhythm generates regular consistent peaks 512 in consecutive two-second windows (for purposes of simplicity, drawing shows one window only).

FIG. 5B shows a two-second window 520 with a rectified and filtered subcutaneous ventricular fibrillation signal 522. The auto-adjusting threshold waveform is also shown at 524. Rising edge crossings between the subcutaneous signal and the auto-adjusting threshold are depicted at 526. Histograms of the signal crossings are shown for a two-second window at 530. Note that ventricular fibrillation generates a gaussian (i.e., bell shaped) distribution 532 centered around 100 mSec in consecutive two-second windows.

FIG. 5C shows a two-second window 540 with a rectified and filtered physiologic noise signal (i.e., myopotentials) 542. The auto-adjusting threshold waveform is also shown at 544. Rising edge crossings between the subcutaneous signal and the auto-adjusting threshold are depicted at 546. Histograms of the signal crossings are shown for a two-second window at 550. Note that noise signals generates high frequency occurrence 552 of short interval crossings (range 15-75 mSec) in consecutive two-second windows.

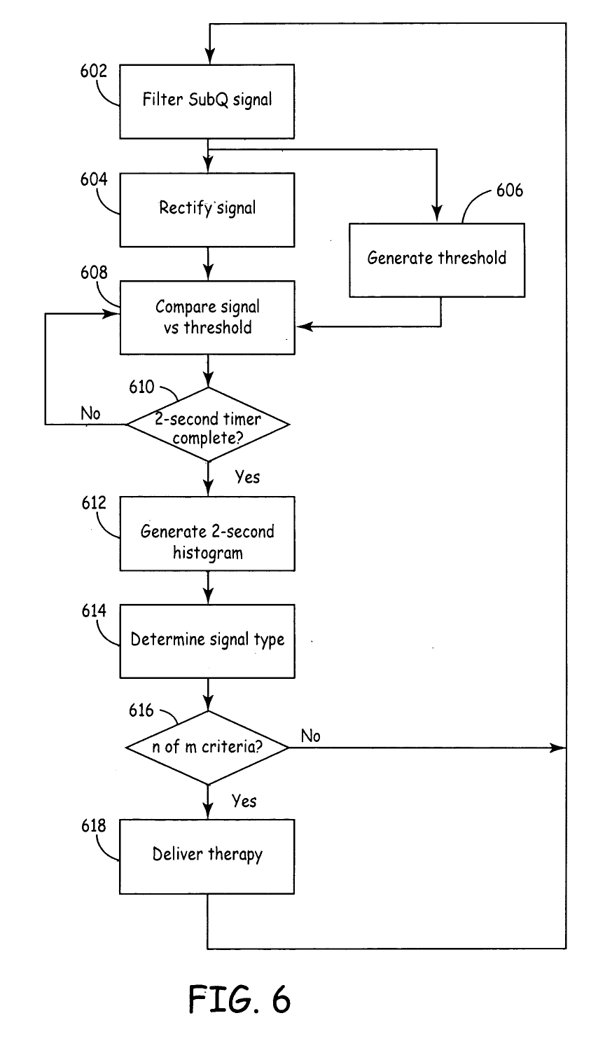

FIG. 6 is a simplified flow diagram 600 illustrating a process of detection of arrhythmias in accordance with the present invention. At step 602 the subcutaneous signal from lead 28 and canister 14 or from bilpolar electrodes on canister periphery is bandpass filtered (0.67-30 Hz) and full wave rectified at step 604. A threshold signal is generated at step 606. This threshold may be programmed/selected from a constant value (percentage of peak value), a moving average and an auto-adjusting threshold value. At step 610 a two-second timer is evaluated. The duration of this timer corresponds to the desired window size and is set to two-seconds for this example. As noted above, a window size between 2 and 10 seconds is desired. If the two-second timer has not completed its timeout, the flow diagram continues to evaluate/compare rectified signals versus the threshold signal/level. If at step 610, the two-second timer is completed, a two-second histogram is generated at step 612. The histogram is evaluated at step 614 and a determination is made whether the subcutaneous signals presented are normal sinus rhythm (regular consistent peaks at longer intervals), ventricular fibrillation (bell shaped gaussian curve near 100 mSec) or noise (high frequency occurrence of short intervals such as 15-75 mSec). A reconfirmation of diagnosis is made at step 616 by considering several adjacent histograms and comparing to a programmable criteria, n similar diagnosis of m adjacent histograms (i.e., 2 of 3). If the diagnosis is not confirmed based on the criteria, the flow diagram returns to step 602 and repeats the signal evaluation process as described hereinabove. If at step 616, the n of m test is affirmatively confirmed, then a pre-programmed therapy is delivered at step 618, as required. After therapy is delivered at step 618, the flow diagram returns to step 602.

It will be apparent from the foregoing that while particular embodiments of the invention have been illustrated and described, various modifications can be made without departing from the spirit and scope of the invention. Accordingly, it is not intended that the invention be limited, except as by the appended claims.

1. A SubQ ICD electrically coupled to at least one subcutaneous electrode forming a subcutaneous sensing and detection system including a level crossing detector, the detector comprising:
means for detecting subcutaneous or body surface signals; and
means for determining the type of signal based on said body surface signals to deliver at least one of pacing, cardioversion and defibrillation therapies transthoracically.

2. The detector of claim 1 wherein said means for determining further includes means for distinguishing between sinus rhythm and ventricular fibrillation from ECG signals colleted via said subcutaneous electrodes.

3. The detector of claim 1 wherein said means for determining includes a bandpass filter and a rectifier.

4. A SubQ ICD comprising:
a subcutaneous electrode;
a housing having a curved surface;
a cardioversion/defibrillation circuit located within the housing and having an electrical connection with the electrode.

5. The SubQ ICD of claim 4 wherein said electrode includes means for detecting subcutaneous body surface signals.

6. The SubQ ICD of claim 4 wherein said housing includes means for detecting subcutaneous body surface signals.

7. The SubQ ICD of claim 4 wherein said circuit includes means for determining the type of signals based on body surface signals to deliver at least one pacing, cardioversion and defibrillation therapies transthoracically.

8. The SubQ ICD of claim 4 wherein said circuit comprises:
means for amplifying and filtering signals from an electrode on said housing and said electrode;
means for rectifying after amplification of said signals; and
means for deriving a level waveform from a narrow based signal.

9. The SubQ ICD of claim 8 wherein a computer determines signal crossings from a selected and generated threshold.

10. The SubQ ICD of claim 4 wherein a microprocessor, control circuit and a software program implemented therein cooperates to generate a histogram with 10 ms bins.

11. The SubQ ICD of claim 10 wherein one or more histograms from adjacent windows are evaluated for detection of one and combination of normal sinus rhythm, physiologic noise signals, sinus tachycardia, ventricular tachycardia and/or ventricular fibrillation.

12. The SubQ ICD of claim 11 wherein the window size is approximately between 2 and 10 seconds.

13. The SubQ ICD of claim 11 wherein a programmable n of m (2 of 3) criteria is implemented to make a determination of VF or CT versus non-life threatening signals.

14. A system of detecting noise, sinus rhythm and ventricular fibrillation based on signals collected from subcutaneous electrodes associated with a SubQ ICD, the system comprising:
a detection circuit; and
a computer implemented software system in operable data communication with the detection circuit.

15. The system of claim 14 wherein said circuit includes:
a filter for filtering the signals collected from said subcutaneous electrodes;
a rectifier to rectify the signals;
a comparator to compare rectified signals against a threshold;
a timer to set a time limit;
a generator for generating a histogram based on the set time limit; and
a determinant for identifying the signal type.

16. The system of claim 15 wherein an n of m criteria is implemented by the software system subsequent to the determinant identifying the signal type.

17. The system of claim 16 wherein therapy is delivered subsequent to n of m criteria is implemented.

18. The system of claim 14 wherein the bandpass filtered range is between about 0.67 to 30 Hz.

19. The system of claim 14 wherein the computer implemented software system processes signals versus threshold crossings and discriminates between VTNF, normal sinus rhythm and noise.

20. The system of claim 19 wherein normal sinus rhythm show regular consistent peaks at wider intervals, VF gaussian distribution is around cycle length near 75-150 mSec and noise exhibits layer number of crossing and lower intervals and is around cycle length near 15-75 mSec.
